(19) United States
(12) Patent Application Publication
(10) Pub. No.: US 2025/0375317 A1
(43) Pub. Date: Dec. 11, 2025

(54) OCULAR PROTECTION MASK

(71) Applicant: SKIS ROSSIGNOL - CLUB ROSSIGNOL, Saint-Jean-de-Moirans (FR)

(72) Inventor: Thomas Morvan, Le Gua (FR)

(73) Assignee: SKIS ROSSIGNOL - CLUB ROSSIGNOL, Saint-Jean-de-Moirans (FR)

(21) Appl. No.: 19/228,230

(22) Filed: Jun. 4, 2025

(30) Foreign Application Priority Data
Jun. 10, 2024 (FR) FR2406064

(51) Int. Cl. A61F 9/02

(52) U.S. Cl. CPC A61F 9/025; A61F 9/027

(57) ABSTRACT

The ocular protection mask (1) has a frame (7) and a visor system (14) fixed to the frame, the visor system including a first visor (2) and a support (13) fixed to the first visor, the first visor covering the front of the frame and the support, which includes a front surface (15) fixed against a rear face (16) of the first visor, and a retaining surface (23) extending substantially parallel to the first visor behind the first visor, the retaining surface and an external edge (3) of the first visor together forming a radial groove (22) cooperating with the frame (7) to retain the visor system on the frame.

### CROSS-REFERENCE TO RELATED APPLICATION

This application claims priority of French patent application No. FR2406064 filed Jun. 10, 2024, the content of which is hereby incorporated by reference herein in its entirety.

### TECHNICAL FIELD OF THE INVENTION

The invention concerns an ocular protection mask intended to protect the eyes, such as a ski mask. The ocular protection mask is intended in particular to protect the eyes against the sun's rays and/or against splashes when practising a sporting activity. The invention also relates to an ocular protection mask of “apparently frameless” type, that is to say an ocular protection mask including a visor covering the front of a frame arranged behind the visor.

### BACKGROUND ART

To protect the eyes against the sun's rays and/or splashes when practising an activity it is standard practice to wear an ocular protection mask equipped with a transparent or translucent protection visor. Wearing such a mask is particularly recommended for practising sports on snow such as skiing or snowboarding, but also for off-road cycling, motorcycling and activities during which high-velocity projectiles are used, such as shooting. Such a mask is pressed against the face around the eyes and therefore offers particularly effective ocular protection.

Known in particular are apparently frameless ocular protection masks. Such masks include a protection visor covering the front of a frame arranged behind the visor. Thus seen from in front, that is to say when looking at the wearer of such a mask face-to-face, the frame is completely hidden behind the visor and only the visor can be seen. Such a mask offers a widened field of view and has an original aesthetic appearance. The publication WO2012178049A1 discloses such a mask.

Fixing such a protection visor to the frame is complicated. In particular known prior art fixing means for fixing the protection visor to the frame are relatively bulky. Consequently, to provide sufficient space for these fixing means the protection visor is at a very great distance from the face. This penalises the aesthetic appearance of such masks and their field of view. Furthermore, known prior art fixing means are relatively fragile and can easily be damaged, for example when handling such a mask or if the user suffers a fall. The visor could then become detached from the frame accidentally or unintentionally, exposing the face of the user to the outside environment, or even cause injury.

### SUMMARY OF THE INVENTION

The object of the invention is to provide an ocular protection mask remedying the above disadvantages and improving known prior art ocular protection masks.

To be more precise, a first object of the invention is an apparently frameless ocular protection mask that is particularly robust, compact and simple to manufacture.

The invention relates to an ocular protection mask comprising a frame and a visor system fixed to the frame, the visor system including a first visor and a support fixed to the first visor, the first visor covering the front of the frame and the support, the support including:
a front surface fixed against a rear face of the first visor, and
a retaining surface extending substantially parallel to the first visor behind the first visor,

the retaining surface and an external edge of the first visor together forming a radial groove cooperating with the frame to retain the visor system on the frame.

The support may further include a rear surface extending in front of the frame, the retaining surface extending between the front surface of the support and the rear surface of the support.

The frame may include a radial groove, the retaining surface being inserted in the radial groove.

The retaining surface may extend along at least 50%, preferably at least 80%, of an external edge of the visor.

The retaining surface may include at least one notch and the frame may include at least one protuberance having a shape complementary to the shape of the at least one notch, the at least one protuberance nesting in the at least one notch.

The at least one notch may have a narrowed inlet.

The at least one notch may include:
at least one first notch formed on a portion of the retaining surface intended to extend around the nose,
at least one second notch formed on a portion of the retaining surface intended to extend in front of a cheek, and
at least a third notch formed on a portion of the retaining surface intended to extend in front of the forehead.

The retaining surface may extend at least:
in a lower part of the support intended to extend in front of the cheeks and around the nose,
in an upper part of the support intended to extend in front of the forehead,
in a left lateral part of the support intended to extend opposite an outside edge of the left eye, and
in a right lateral part of the support intended to extend opposite an outside edge of the right eye.

The frame may be more deformable than the visor system, only the frame being intended to be deformed to assemble or to disassemble the visor system onto or from the frame.

The ocular protection mask may include an elastic strap configured to retain the ocular protection mask on the head of a person, the elastic strap being fixed to the frame.

The entirety of the external edge of the first visor may be visible from outside the mask.

The visor system may include a second visor extending parallel to the first visor behind the first visor, the second visor being retained by the support.

### BRIEF DESCRIPTION OF THE DRAWINGS

The above objects, features and advantages of the present invention are described in detail in the following description of one particular embodiment given by way of non-limiting example with reference to the appended figures, in which:

FIG. 1 is a view of the front face of an ocular protection mask in accordance with one embodiment of the invention.

FIG. 2 is a view of the ocular protection mask from above.

FIG. 3 is a side view of the ocular protection mask.

FIG. 4 is a perspective view of a visor support of the ocular protection mask.

FIG. 5 is a view of the rear face of the ocular protection mask.

FIG. 6 is a rear view of the visor system of the ocular protection mask, the visor system comprising the support from FIG. 4 and the visor from FIG. 5.

FIG. 7 is a view in section of a part of the visor system of the ocular protection mask.

FIG. 8 is a view in section of a part of the visor system and of a frame of the ocular protection mask, the frame cooperating with the visor system to retain the visor system.

FIG. 9 is a perspective view of a body of the frame of the ocular protection mask.

FIG. 10 is a detail view of a notch formed in a flange of the visor support of the ocular protection mask.

FIG. 11 is an exploded view of the ocular protection mask, the mask including a first visor and a second visor.

### DETAILED DESCRIPTION OF PARTICULAR EMBODIMENTS

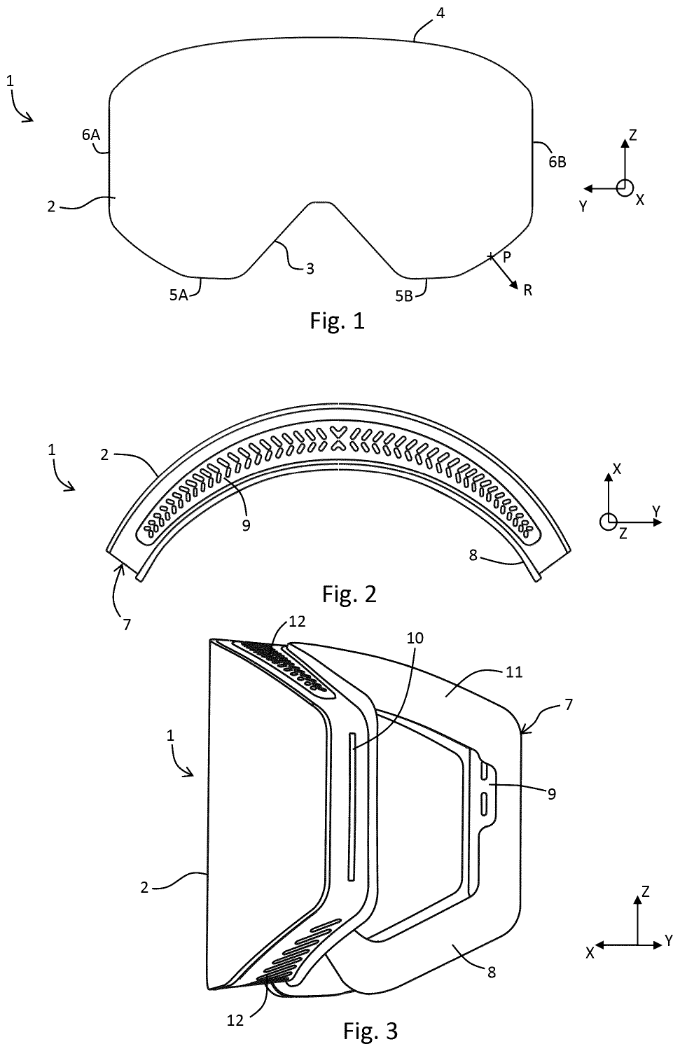

FIG. 1 depicts an ocular protection mask 1 in accordance with one embodiment of the invention. The ocular protection mask 1 is intended to protect the eyes from the sun's rays and/or splashing when practising a sporting activity such as a sport on snow such as alpine skiing or snowboarding, off-road biking, motorcycling, or an activity in which high-speed projectiles are used, such as shooting. When it is used for the practise of a sport on snow such a mask may be called a “ski mask”.

The ocular protection mask 1, simply referred to as “the mask 1” hereinafter, includes a transparent or translucent protection visor 2 through which a user is able to observe the environment. The visor 2 therefore extends in a field of view of a wearer of the mask and notably in front of the two eyes of its user. The visor 2 can in particular be tinted to shade a perception of the environment and thus to protect a wearer of the mask from the sun's rays. Alternatively, the visor 2 may be transparent simply to protect the eyes from splashing without shading the perception of the environment. The visor 2, which may also be referred to as the “glass”, is preferably made of plastic material, in particular of polycarbonate.

The mask 1 is of the “apparently frameless” type. Only the visor 2 is directly visible when observing the mask 1 from the front, that is to say from the FIG. 1 point of view. The mask 1 therefore preferably includes a smooth front face that is not locally covered by any part of the frame or any other structural element intended to retain the visor. The mask 1 therefore does not include hems or borders on its front face at the interface between the visor 2 and such a structural element. This prevents particles from being able to accumulate in such borders. The visor 2 is therefore particularly simple to clean and offers an original aesthetic appearance.

Note that, assuming that the visor 2 is sufficiently transparent, it nevertheless remains possible to distinguish components of the mask 1 other than its visor 2 by looking through the visor. A mask in which a part of the frame is visible, as seen from the front, only through the visor 2 is nevertheless considered an “apparently frameless” mask in the context of the invention.

The mask 1 is intended to be pressed onto the face around the eyes. In the front view represented in FIG. 1 the visor 2 has a broadly rectangular shape with a notch to fit around the nose. The visor 2 has an external edge 3, or contour 3, notably formed by:
an upper edge 4 extending in front of the forehead,
two lower edges 5A, 5B on respective opposite sides of the notch fitting around the nose and extending face-to-face with the cheeks, and
lateral edges 6A, 6B extending substantially vertically at the sides of the eyes.

At least 80% of the external edge 3 of the visor or even at least 90% of the external edge 3 of the visor could preferably be visible. In the embodiment depicted the external edge 3 of the visor 2 is entirely visible from outside the mask 1, that is to say 100% of the external edge 3 of the visor is visible and not covered by a structural element of the mask.

In a view from above depicted in FIG. 2 it is seen that the mask 1 has a rounded shape intended to espouse the shape of the head and the face of the wearer of the mask. Generally speaking, the visor 2 can for example be the shape of a portion of a circular cylinder or even the shape of a portion of a sphere.

The longitudinal axis X is defined as the axis of the view of a wearer of the mask when looking straight ahead. The longitudinal axis is a horizontal axis oriented from the rear toward the front. The vertical axis Z is defined as the axis of a wearer of the mask when standing upright. The vertical axis Z is perpendicular to the longitudinal axis X. The vertical axis Z is oriented upward. Finally, the transverse axis Y is defined as the axis perpendicular to the axes X and Z and oriented from left to right from the point of view of the wearer of the mask 1. The axes X, Y and Z form an orthogonal system of axes. For a given point P on the external edge 3 of the visor 2 the radial axis R is defined as the axis both parallel to the visor 2 at the level of the point P concerned and perpendicular to the external edge 3 at the level of the point P. The radial axis R is oriented in a centrifugal direction.

As can be seen in FIGS. 2 and 3 the mask 1 includes a frame 7 supporting the visor 2. The frame 7 is equipped with an elastic strap (not represented) intended to pass behind the head or behind a helmet to press the mask 1 against the face of its wearer. The elastic strap has a first end fixed to a left-hand edge of the frame 7 and a second end fixed to a right-hand edge of the frame. To be more precise, the frame 7 includes a body 8 and left and right fixing means 9 cooperating with the two ends of the elastic strap. The two fixing means 9 are integrated into the body 8. The elastic strap passes through the body 8 via left and right slots 10 provided for this purpose. The frame 7 advantageously includes a circumferential bearing surface 11 consisting of a flexible material able to be deformed in contact with the face. As explained above, the frame 7 is covered at the front by the visor 2. By “at the front” is meant that the frame 7 is covered by the visor when seen from the front.

The frame 7 advantageously also includes ventilation means 12, for example in the form of one or more grilles fixed to the body 8 or formed directly in the body 8. The ventilation means 12 can for example be arranged at the level of an upper part and/or a lower part of the frame 7, between the visor 2 and the bearing surface 11. The ventilation means 12 are for preventing mist forming on the visor 2.

The body 8 of the frame 7 advantageously consists of a flexible elastic material. The bearing surface 11 can therefore be formed directly on the body 8. The body 8 can therefore be intended to bear directly against the face. Alternatively, the bearing surface 11 can be formed on an element distinct from the body 8 and mounted on, for example glued to, the body 8. Such a mounted element can for example be a foam or woven element.

In one embodiment the body 8 of the frame can consist of thermoplastic polyurethane (TPU). The material constituting the body can have a Shore A hardness as measured in accordance with the standard ASTM D2240 between 80 and 90.

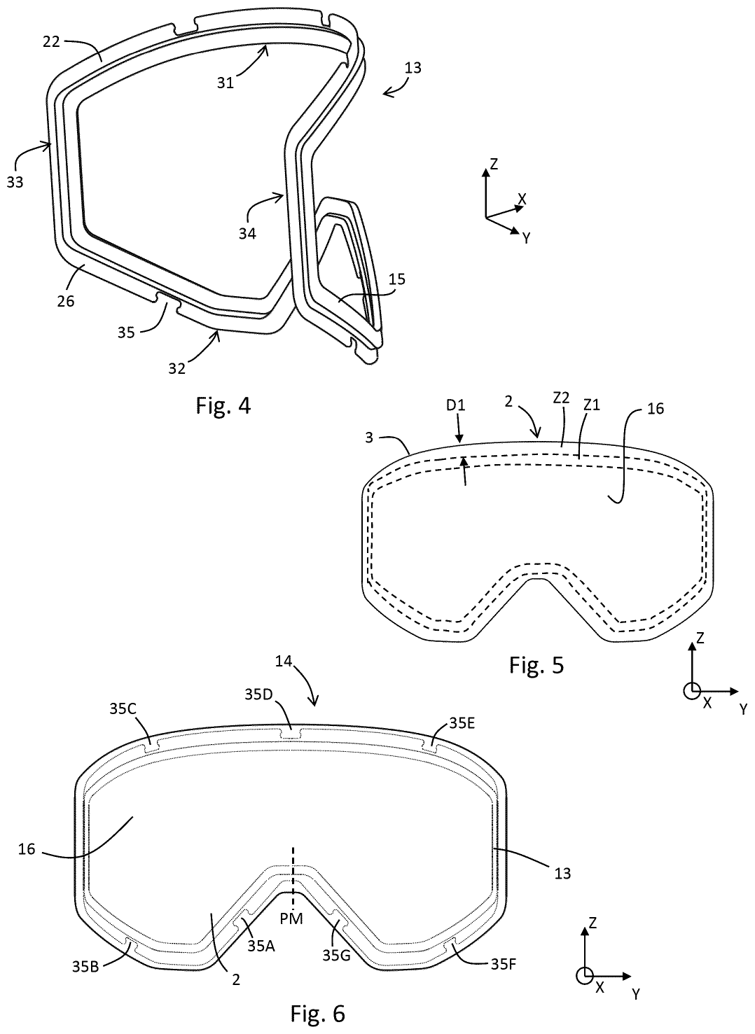

Described next with reference to FIGS. 4 to 10 is a means for fixing the visor 2 to the frame 7 in one embodiment of the invention. The visor 2 is fixed to the frame 7 by means of a visor support 13, simply referred to as “the support 13” hereinafter. The support 13 is depicted in FIG. 4. The visor 2 is depicted in FIG. 5. The “visor system 14” is the assembly formed by the visor 2 and the support 13. The visor system 14 is depicted in FIG. 6. In particular the visor 2 is fixed onto an external face of the support 13. The visor 2 can therefore be referred to as the external visor.

The visor 2 covers the front not only of the frame 7 as explained above but also the support 13. The support 13 therefore does not project radially from the external edge 3 of the visor. The support 13 is therefore invisible when seen from in front from outside the mask 1 or visible only through the visor 2. As emerges hereinafter the support 13 is also invisible when observing the mask 1 from the side because it is covered by the frame 7. The support 13 has a general shape that globally espouses the shape of the external edge 3 of the visor 2. The support 13 includes in particular a closed line delimiting a central opening closed by the visor 2. In the embodiment shown the line that follows the support 13 is uninterrupted and therefore follows the entirety of the external edge 3 of the visor. In a variant the support could extend along only part of the external edge 3 of the visor 2, preferably over at least 50% of the length of the external edge 3 or even at least 80% of the length of the external edge 3. In another variant the support 13 could be formed of a plurality of support elements separated from one another along the external edge 3 of the visor 2.

The support 13 is preferably a one-piece element, preferably made of plastic, for example a moulded plastic element. The support 3 can consist of a thermoplastic polymer such as acrylonitrile butadiene styrene (ABS), polycarbonate (PC) or a mixture of these two materials.

The support 13 is fixed to visor 2. In particular, the support 13 includes a front surface 15 fixed against a rear face 16 of the visor 2. The front surface 15 of the support 13 is preferably glued against the rear face 16 of the visor 2. In one variant the support 13 and the visor 2 could be fixed together differently, for example by nesting or welding. Alternatively, the support 13 and the visor 2 could even form a single one-piece part.

A first zone Z1 of the rear face 16 of the visor 2 against which the front surface 15 of the support 13 is fixed is identified by dashed lines in FIG. 5. This first zone Z1 is distant from the external edge 3 of the visor 2 at a non-zero distance D1, for example at least 5 mm. The rear face 16 includes a second zone Z2 between the first zone Z1 and the external edge 3 of the visor 2. The second zone therefore has a non-zero width. The second zone Z2 of the visor bears directly against the frame 7, in particular against the body 8 of the frame 7. The second zone Z2 of the visor 2 therefore masks the frame 7 when the mask 1 is seen from the front. In other words the second zone Z2 of the visor covers an external face of the frame at least in part.

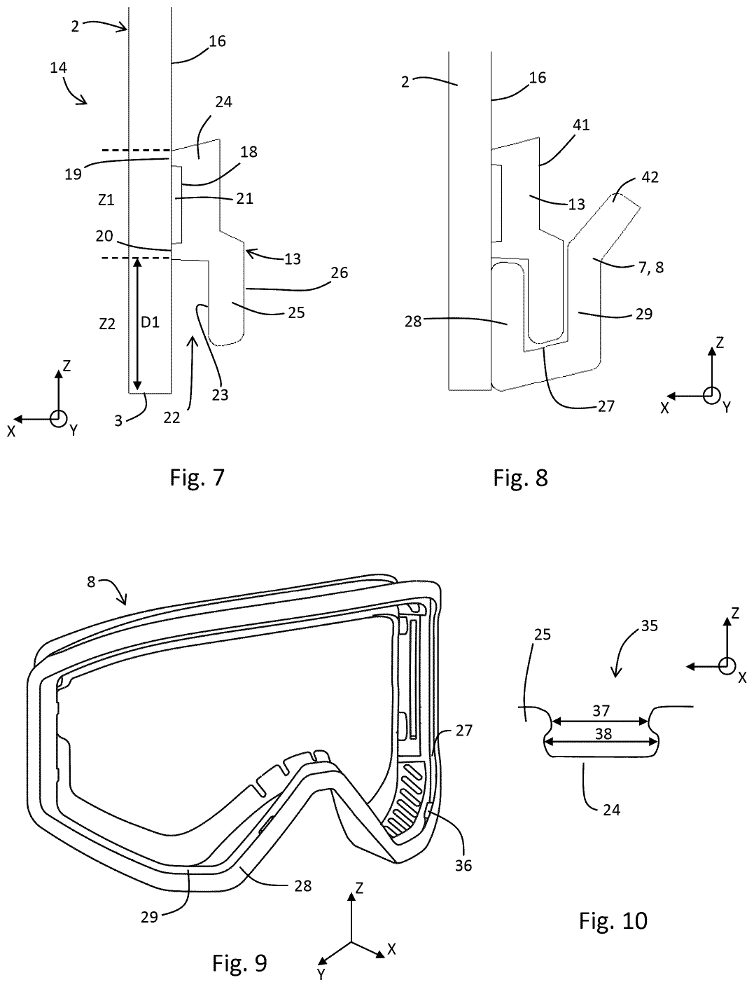

FIG. 7 depicts by a view in section on a median plane PM a part of the visor system 14. The median plane PM is identified in FIG. 6. It is a plane parallel to the axes X and Z passing through a centre of the mask 1. It is seen that the front surface 15 of the support 13 includes a set back zone 18 framed by an internal rim 19 and an external rim 20. The rims 19 and 20 bear directly against the rear face 16 of the visor 2. The set back zone 18 enables a free volume 21 to be formed between the rear face 16 of the visor 2 and the support 13. This free volume 21 is intended to receive glue for fixing the visor 2 onto the support 13. This makes it possible to circumscribe the surface of the visor receiving the glue and therefore to prevent the glue leaking onto a useful zone of the visor. The rims 19 and 20 advantageously bear against the visor over the entire perimeter of the visor without interruption. The bead of glue for fixing the support 13 to the visor 2 therefore follows a closed and continuous line. This improves the retention of the support 13 on the visor 2. Alternatively a self-adhesive tape, for example a self-adhesive seal, could be disposed in the set back zone 18, between the visor 2 and the support 13. In a variant the front surface 15 could include discontinuities. Note that in order to mask the support 13 and the frame 7 the visor 2 can advantageously be tinted at least at the level of the zones Z1 and Z2.

The support 13 also includes a retaining surface 23 extending parallel to the front surface 15 and offset rearwardly relative to the front surface 15. The retaining surface 23 also extends parallel to the visor 2. The retaining surface 23 cooperates with the frame 7, in particular with the body 8 of the frame 7, to retain the visor system 14 on the frame. In particular, a reaction force of the retaining surface 23 against the body 8 of the frame 7 prevents frontal separation of the visor 14 from the frame 7.

The retaining surface 23 extends farther to the rear than the front surface 15. A radial groove 22 is therefore formed between the retaining surface 23 of the support 13 and the external edge 3 of the visor 2. As emerges hereinafter the body 8 includes a front wall 28 inserted in this radial groove 22. Note that the branch of the radial groove 22 formed by the visor 2 is higher than the branch of the radial groove 22 formed by the support 13. This enables the visor 2 to cover efficaciously not only the support 13 but also the frame 7.

In another approach the support 13 includes a base 24 fixed to the visor and a flange 25 extending from the base 24 parallel to the visor 2 in the direction of the external edge 3 of the visor. The flange 25 therefore extends radially in a centrifugal direction.

The base 24 includes the front surface 15 of the support and the flange 25 includes the retaining surface 23.

The retaining surface 23 extends along the external edge 3 of the visor, preferably along at least 50% of the length of the external edge 3 of the visor, or even at least 80% of the length of the external edge 3 of the visor. The farther the retaining surface 23 extends along the external edge 3 of the visor the better the retention conferred by the cooperation of that retaining surface with the frame 7. In one embodiment the retaining surface 23 extends 360° around the visor 2. A retaining surface extending along a high proportion of the external edge 3 of the visor therefore makes it possible to envisage a retaining surface of low height along the radial axis R and/or the use of a more flexible and therefore more comfortable material to produce the body 8 of the frame 7.

The retaining surface 23 preferably extends at least:
in a lower part 31 of the support intended to extend in front of the cheeks and around the nose of the wearer of the mask 1,
in an upper part 32 of the support intended to extend in front of the forehead of the wearer of the mask 1,
in a left lateral part 33 of the support intended to extend in front of an exterior side of the left eye on the forehead of the wearer of the mask 1, and
in a right lateral part 34 of the support intended to extend in front of an exterior side of the right eye on the forehead of the wearer of the mask 1. The visor system 14 is therefore retained on the frame 7 on its various sides, which makes it possible to obtain homogeneous and robust retention of the visor system 14.

The support 13 also has a rear surface 26 extending in front of the frame 7, in particular in front of the body 8 of the frame 7. The rear surface 26 extends parallel to the visor 2. The rear surface 26 can bear at least locally against the frame 7. The rear surface 26 can also extend at least locally at a non-zero distance from the frame, as can be seen in FIG. 8. The distance D2 separating the front surface 15 from the rear surface 26 of the support can preferably be less than or equal to 5 mm, or even less than or equal to 3 mm. The support 13 is therefore particularly compact along the longitudinal axis X. The rear surface 26 and the retaining surface 23 of the support are two opposite faces of the flange 25. The rear surface 26 faces the back of the mask 1 and the retaining surface 23 faces the front. The rear surface 26 is globally plane. A large contact area is therefore obtained between the frame 7 and the support 13 and there is no risk of the support 13 being damaged in the event of an axial force being exerted on the visor 2.

As can be seen in FIGS. 8 and 9 the body 8 of the frame 7 includes a radial groove 27. The radial groove 27 extends parallel to the radial axis R over the entire perimeter of the frame 7. This radial groove 27 is formed by a front wall 28 and a rear wall 29 extending parallel to the front wall 28. The flange 25 of the support 13 is inserted in the radial groove 27 of the frame. The support 13 is therefore masked by the frame 7 at the sides of the mask 1. The retaining surface 23 of the support being formed on the flange 25, the retaining surface 23 is therefore also inserted in the radial groove 27 of the frame.

A front face of the front wall 28 of the frame bears against or can bear against the second zone Z2 of the rear face 16 of the visor 2. A rear face of the front wall 28 bears against or can bear against the retaining surface 23 of the support. The thickness of the front wall 28 is advantageously substantially equal to the distance separating the retaining surface 23 of the support from the rear face 16 of the visor. A front face of the rear wall 29 bears against or can bear against the rear surface 26 of the support. The radial groove 27 advantageously has a constant width at the perimeter of the frame 7.

Referring to FIGS. 4, 6 and 9, it is seen that the retaining surface 23 of the support includes a set of notches 35. The body 8 of the frame 7 includes a set of protuberances 36 with a shape complementary to the shape of the notches 35. Each protuberance 36 of the frame is therefore nested in a notch 35 of the support. This makes it possible to improve the retention of the visor system 14 on the frame 7. The notches 35 are local interruptions of the retaining surface 23 and even the only interruptions of the retaining surface 23. The protuberances 36 are arranged at the bottom of the radial groove 27, in particular between the front wall 28 and the rear wall 29 of the frame.

All the notches 35 can have identical shapes and all the protuberances 36 can have identical shapes. In particular, as depicted in FIG. 10, each notch 35 advantageously includes a narrowed inlet 37, that is to say an inlet 37 narrower than the bottom 38 of the notch 35. Engaging the protuberance 36 inside the notch 35 therefore requires elastic deformation of the protuberance 36 and/or of the notch 35. Such an artifice further improves the retention of the visor system 14 on the frame 7 without penalising the overall size of the mask 1.

In the embodiment shown the retaining surface 23 includes seven notches 35 distant from one another. Four notches are formed in the lower part 31 of the support and three notches are formed in the upper part 32 of the support.

To be more precise, the support 13 includes:
a first notch 35A formed on a portion of the retaining surface intended to extend around the nose, in particular on the right-hand side of the nose of the wearer of the mask,
a second notch 35B formed on a portion of the retaining surface intended to extend in front of the right cheek of the wearer of the mask,
a third notch 35C formed on a portion of the retaining surface intended to extend in front of the forehead, in particular substantially above the right eye of the wearer of the mask,
a fourth notch 35D formed on a portion of the retaining surface intended to extend in front of the forehead, in particular above the nose,
a fifth notch 35E formed on a portion of the retaining surface intended to extend in front of the forehead, in particular substantially above the left eye of the wearer of the mask,
a sixth notch 35F formed on a portion of the retaining surface intended to extend in front of the left cheek of the wearer of the mask, and
a seventh notch 35G formed on a portion of the retaining surface intended to extend around the nose, in particular on the left-hand side of the nose of the wearer of the mask.

The body 8 includes seven protuberances 36 positioned in front of the seven notches.

Alternatively the number of notches 35 and the number of corresponding protuberances 36 could be different. The mask preferably comprises between five and ten notches and as many corresponding protuberances so as to ensure good retention of the visor system 14 on the frame 7 while maintaining simple assembly of these two elements.

The visor system 14 is advantageously attached to the frame 7 in a removable manner, i.e. reversibly. In particular the visor system 14 can be assembled and disassembled onto and from the frame 7 by the user of the mask 1 without using tools. The mask 1 can advantageously be delivered with several visor systems 14 of identical shape. Such visor systems can differ from one another by the opacity of their visor (for example to adapt to different climatic or visibility conditions) and/or by their aesthetic appearance, or can even be identical to each other so as to serve as spares. Visor systems 14 can also be marketed independently of the frame 7 to allow users of the goggles to replace their visor.

The body 8 of the frame 7 is advantageously more deformable than the visor system 14. The body 8 of the frame 7 may in particular be made of a plastic material that is less hard than the plastic material constituting the support 13. The visor system 14 may be considered a non-deformable assembly compared to the body of the frame. Thus only the frame 7 is intended to deform to assemble or disassemble the visor system onto or from the frame. The flexibility of the body 8 of the frame 7 is therefore used both to provide comfortable support for the frame 7 on the face and to allow assembly and/or disassembly of the visor system 14. For example the body 8 can have a tensile strength less than or equal to 40 MPa according to the ASTM D412 standard. The support 13 can have a tensile strength greater than or equal to 50 MPa according to the ASTM D638 standard.

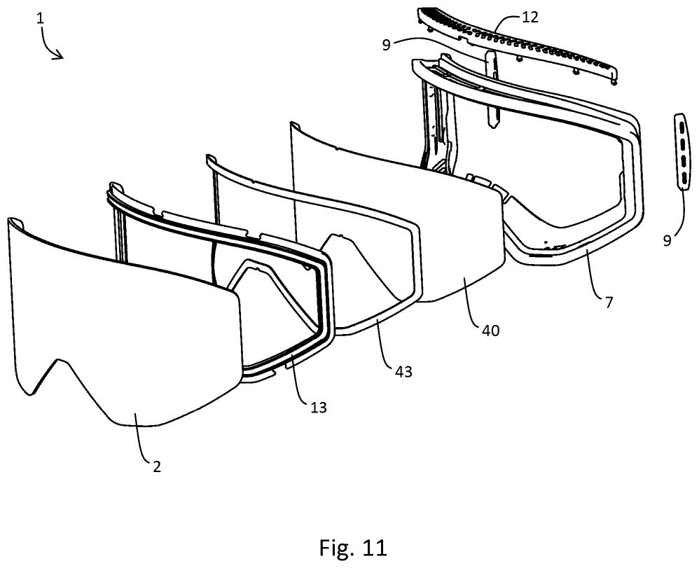

FIG. 11 depicts an embodiment in which the mask 1 includes a second visor 40. The second visor 40 can be fixed to the support 13 behind the visor 2 described above referred to as “the first visor 2”. The second visor 40 preferably extends parallel to the first visor 2 at a non-zero distance from the first visor 2. A layer of air is therefore formed between the first visor and the second visor. This layer of air serves as a heat barrier and makes it possible to prevent the formation of mist inside the mask 1. Thus the first visor 2 constitutes a front visor and the second visor 40 constitutes a rear visor.

The support 13 advantageously includes a bearing surface 41 (identified in FIG. 8) extending over a rear perimeter of the support 13 and against which the second visor 40 is fixed, for example glued. The bearing surface 41 is formed on a rear face of the base 24. The bearing surface 41 is offset toward the front relative to the rear surface 26 described above. A rear face of the second visor 40 is therefore able to extend substantially in line with the rear surface 26. The body 8 of the frame can optionally include a tongue 42 covering an external edge of the second visor. The second visor 40 can be fixed to the support 13 by means of glue or by means of adhesive tape 43, such as a self-adhesive seal as depicted in FIG. 11. The support 13 therefore serves to retain not only the first visor 2 but also the second visor 40. The second visor 40 has an area strictly less than the area of the first visor 2. The first visor 2 extends radially farther than the second visor 40.

Finally, thanks to the invention an ocular protection mask 1 is obtained that is simple to manufacture and to use. The means for fixing the visor system 14 to the frame 7 extend radially and are therefore particularly compact in the axial direction. This makes it possible to retain a visor 2 close to the face and a wide field of view.

1. An ocular protection mask comprising:
a frame, and
a visor system fixed to the frame,
the visor system including a first visor and a support fixed to the first visor, the first visor covering the front of the frame and the support, wherein the support includes:
a front surface fixed against a rear face of the first visor, and
a retaining surface extending substantially parallel to the first visor behind the first visor,
the retaining surface and an external edge of the first visor together forming a radial groove cooperating with the frame to retain the visor system on the frame.

2. The ocular protection mask according to claim 1, wherein the support further includes a rear surface extending in front of the frame, the retaining surface extending between the front surface of the support and the rear surface of the support.

3. The ocular protection mask according to claim 1, wherein the frame includes a radial groove, the retaining surface being inserted in the radial groove.

4. The ocular protection mask according to claim 1, wherein the retaining surface extends along at least 50% of an external edge of the visor.

5. The ocular protection mask according to claim 1, wherein the retaining surface includes at least one notch and the frame includes at least one protuberance having a shape complementary to a shape of the at least one notch, the at least one protuberance nesting in the at least one notch.

6. The ocular protection mask according to the claim 5, wherein the at least one notch has a narrowed inlet.

7. The ocular protection mask according to claim 4, wherein the at least one notch includes:
at least one first notch formed on a portion of the retaining surface intended to extend around a nose,
at least one second notch formed on a portion of the retaining surface intended to extend in front of a cheek, and
at least a third notch formed on a portion of the retaining surface intended to extend in front of a forehead.

8. The ocular protection mask according to claim 1, wherein the retaining surface extends at least:
in a lower part of the support intended to extend in front of cheeks and around a nose,
in an upper part of the support intended to extend in front of a forehead,
in a left lateral part of the support intended to extend opposite an outside edge of a left eye, and
in a right lateral part of the support intended to extend opposite an outside edge of a right eye.

9. The ocular protection mask according to claim 1, wherein the frame is more deformable than the visor system, only the frame being intended to be deformed to assemble or to disassemble the visor system onto or from the frame.

10. The ocular protection mask according to claim 1, wherein the ocular protection mask includes an elastic strap configured to retain the ocular protection mask on a head of a person, the elastic strap being fixed to the frame.

11. The ocular protection mask according to claim 1, wherein an entirety of an external edge of the first visor is visible from outside the mask.

12. The ocular protection mask according to claim 1, wherein the visor system includes a second visor extending parallel to the first visor behind the first visor, the second visor being retained by the support .

13. The ocular protection mask according to claim 2, wherein the frame includes a radial groove, the retaining surface being inserted in the radial groove.

14. The ocular protection mask according to claim 2, wherein the retaining surface extends along at least 50% of an external edge of the visor.

15. The ocular protection mask according to claim 3, wherein the retaining surface extends along at least 50% of an external edge of the visor.

16. The ocular protection mask according to claim 1, wherein the retaining surface extends along at least 80% of an external edge of the visor.

17. The ocular protection mask according to claim 2, wherein the retaining surface extends along at least 80% of an external edge of the visor.

18. The ocular protection mask according to claim 3, wherein the retaining surface extends along at least 80% of an external edge of the visor.

19. The ocular protection mask according to claim 2, wherein the retaining surface includes at least one notch and the frame includes at least one protuberance having a shape complementary to a shape of the at least one notch, the at least one protuberance nesting in the at least one notch.

20. The ocular protection mask according to claim 19, wherein the at least one notch has a narrowed inlet.
